(19) United States
(12) Patent Application Publication
(10) Pub. No.: US 2012/0252730 A1
(43) Pub. Date: Oct. 4, 2012

(54) PLATELET AGGREGATION INHIBITORS

(75) Inventors: Milan Mrksich, Hinsdale, IL (US); Juan Sanchez-Cortes, Chicago, IL (US)

(73) Assignee: UNIVERSITY OF CHICAGO, Chicago, IL (US)

(21) Appl. No.: 13/393,608

(22) PCT Filed: Sep. 2, 2010
(86) PCT No.: PCT/US10/47695

§ 371 Date: Jun. 19, 2012

Related U.S. Application Data
(60) Provisional application No. 61/239,499, filed on Sep. 3, 2009.

(51) Int. Cl. C07K 7/64; A61P 7/02; C07K 5/08; A61K 38/12; A61K 38/08; C07K 7/06; A61P 7/04; A61P 9/00

(52) U.S. Cl. 514/13.7; 514/13.8; 514/16.4; 514/21.1; 514/21.7; 530/317; 530/329

(57) ABSTRACT

Peptides and cyclized analogs thereof that are useful as platelet aggregation inhibitors in the treatment of cardiac disease, including acute coronary syndrome are disclosed.

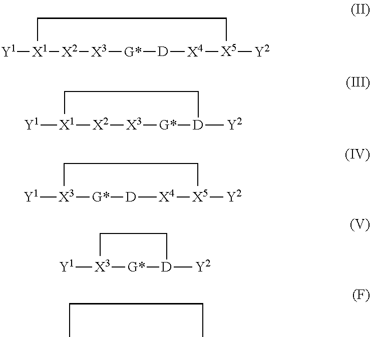

### CROSS REFERENCE TO RELATED APPLICATIONS

This application claims the benefit of U.S. Provisional Patent Application No. 61/239,499 filed Sep. 3, 2009, the content of which is hereby incorporated by reference in its entirety.

### FEDERALLY SPONSORED RESEARCH AND DEVELOPMENT

This invention was made with government support under CCNE NIH Award Number U54CA119341 awarded by National Institutes of Health (NIH). The government has certain rights in the invention.

### BACKGROUND

Fibrinogen (Fbg) is an abundant plasma protein that is essential for homeostasis. This protein is a disulfide-linked homodimeric complex assembled from α, β and γ subunits and presents multiple peptide motifs that bind the αIIbβ3 integrin receptor present on platelets and αvβ3 on endothelial cells. This way Fbg can aggregate platelets and localize clots to activated endothelium. Fbg also serves as an extracellular matrix protein to mediate cell adhesion following its conversion to insoluble fibrin by the protease thrombin (Bini et al., 2000). Consequently, a substantial effort has been directed towards identifying the binding sequences in Fbg that mediate platelet aggregation and adhesion, and in understanding the differential roles of these ligands. Previously two sequences have been implicated for platelet aggregation—the RGD site on the α subunit and a carboxy-terminal peptide on the γ subunit—yet the mechanistic roles of the two peptides remain controversial. Fbg contains two peptide motifs that are important for its ability to aggregate platelet receptors: an RGD sequence at position 572-574 on the α chain and a HHLGGAKQAGDV (SEQ ID NO: 1) sequence at position 400-411 of the γ chain. There is a second RGD site at position 95 in the α chain, but this ligand is likely conformationally masked within a coiled-coil domain and does not participate in the initial aggregation of platelets (Doolittle et al., 1978, Ugarova et al., 1993). A consensus has emerged that the RGD sequence is important for binding to the αvβ3 receptor on endothelial cells and thereby serves to localize a thrombus to regions of activated endothelium. Further, a series of studies has established that the γ peptide interacts with the platelet receptor and is important for fibrinogen-mediated aggregation of platelets (Hawiger et al., 1982, Kloczewiak, 1984, Farrell et al., 1992). What has been less clear is to what extent the RGD motif is also important in platelet aggregation and whether the γ and RGD peptides bind to common or separate sites on the receptor.

Previous research that supported a model wherein the two peptides bind to non-overlapping sites on the receptor used two monoclonal antibodies to probe the interaction of the receptor with the ligands: PAC-1, which competes with Fbg in binding to αIIbβ3, and A2A9, which binds the integrin at a different site than does PAC-1 and sterically blocks the binding of Fbg to the receptor. The peptide RGDS (SEQ ID NO: 2) blocked the binding of both PAC-1 and Fbg to platelets with equal potency. The γ-derived peptide LGGAKQAGDV (SEQ ID NO: 3) also inhibited Fbg binding to platelets with an affinity comparable to that of RGDS (SEQ ID NO: 2), but was 2.5-fold less potent in inhibiting PAC-1 binding to αIIbβ3. Finally, LGGAKQAGDV (SEQ ID NO: 3), but not RGD, could inhibit the binding of A2A9 to platelets. These results suggest that the two peptides interact with the integrin at two different sites (Bennett et al., 1988). Another study of cross-linking the complex suggested that GRGDS (SEQ ID NO: 4) interacts with the β3 subunit (D'Souza et al., 1988) while HHLGGAKQAGDV (SEQ ID NO: 1) interacts with the heavy chain in the αIIb subunit, giving further evidence in support of two-binding site model (D'Souza et al., 1990). Further support for this model came from studies that used surface plasmon resonance experiments to show that the two binding sites in ΕIIbβ3 are allosterically related (Hu et al., 1999) and a report that the peptides served two distinct functions, with the initial cell adhesion mediated by HHLGGAKQAGDV (SEQ ID NO: 1), and subsequent cell spreading mediated by GRGDS (SEQ ID NO: 4) (Salsmann et al., 2005).

Several cyclic peptides found in snake venoms have been characterized as potent anti-thrombotic agents and operate by using either a RGD or KGD motif to bind the integrin (Scarborough et al., 1993; Oshiwaka et al., 1999). The cyclic peptide eptifibatide—which has a homoarginine residue in the first position—was derived from barbourin (Scarborough et al. 1991) and is now used in clinical settings to prevent platelet aggregation. Eptifibatide has gained widespread use, as it overcomes the immunogenicity and clearance time problems faced by abciximab, a Fab fragment used to inhibit platelet aggregation. Although eptifibatide does not cause immune response and follows renal body clearance (Scarborough, 1999), one of its secondary effects is prolonged bleeding in patients (Curran et al, 2005). Eptifibatide also binds the αvβ3 integrin on endothelial cells with lower affinity, and it has been suggested that the peptide binds this integrin at therapeutic concentrations, though the consequences of this interaction are not yet well characterized (Lele et al., 2001). In this respect, inhibitors that display a greater specificity for αIIb3 relative to other integrins may be valuable. Therefore, there is a need in the art for methods and compounds relating to platelet aggregation inhibitors for the treatment of cardiovascular disorders and other diseases.

### SUMMARY OF THE INVENTION

In one aspect, the present invention provides for compounds of formulas I, II, III, IV, or V:

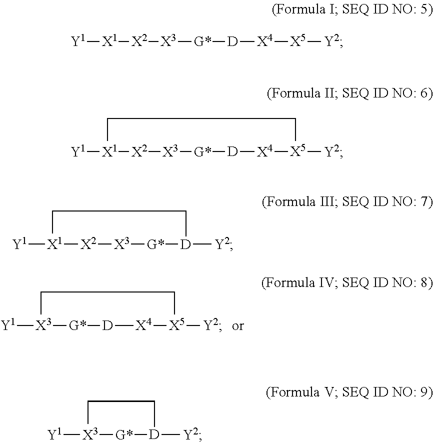

wherein

[symbol]

represents a bond;
X¹ and X⁵ are independently selected residues capable of forming a bond between X¹ and X⁵ to obtain a cyclic compound;
X² is absent or is a sequence of independently selected amino acids that is from one to six amino acids in length;
X⁴ is absent or is a sequence of independently selected amino acids that is from one to six amino acids in length;
wherein the sum of the length of amino acids in X² plus X⁴ is from zero to 6 amino acids;
X³ is a hydrophobic amino acid residue; and
G* is Gly or sarcosyl;
Y¹ is absent, H, acetyl, or an amino protecting group;
Y² is —OH, NH₂, or a carboxyl protecting group;
wherein one or more peptide linkages of Formulas I-V may optionally be replaced by a linkage selected from the group consisting of —CH₂NH—, CH₂CH₂—, —CH═CH— (cis and trans), —COCH₂—, —CH(OH)CH₂— and —CH₂SO—; or a pharmaceutically acceptable salt thereof;
with the proviso that the following peptides are excluded from the scope of formula (I):

(SEQ ID NO: 1)
HHLGGAKQAGDV,
(SEQ ID NO: 3)
LGGAKQAGDV,
and
(SEQ ID NO: 10)
LQAGDV.

In certain embodiments, compounds of formulas I-V may display a greater ability to inhibit the binding of Fbg to αIIbβ3 integrins compared to αvβ3 integrin.

### BRIEF DESCRIPTION OF THE DRAWINGS

FIG. 1 depicts self-assembled monolayers (“SAM”) and their characterization by SAMDI-MS (“self-assembled monolayers for matrix assisted laser desorption ionization mass spectrometry”). (A) Self-assembled monolayer presenting a maleimide group among tri(ethylene glycol) background. The maleimide group allows for the covalent immobilization of Cys-terminated peptides through a Michael addition. The glycol groups prevent nonspecific cell adhesion. (B) SAMDI characterization of a 0.5% GRGDSC (SEQ ID NO: 11) SAM. Tri(ethylene glycol) disulfide and GRGDSC (SEG ID NO: 11)-terminated mixed disulfide were detected. All were sodium salts. (C) SAMDI characterization of a 0.5% HHLGGAKQAGDVC (SEQ ID NO: 21) SAM. Tri(ethylene glycol) disulfide and HHLGGAKQAGDVC (SEQ ID NO: 21)-terminated mixed disulfide were detected. All were sodium salts.

FIG. 2 depicts the results of FACS analyses and binding studies of a αIIbβ3 expressing CHO K1 cell line. In panel A it is shown that CHO K1 cells do not express αIIbβ3 (top) (the middle and right hand lanes show anti-β3 antibody and PAC-1 antibody binding, respectively). Transfected αIIbβ3 CHO K1 cells express αIIbβ3 at the cell surface, as detected by anti-β3 and PAC-1 antibody (bottom) (the middle and right hand lanes show anti-β3 antibody and PAC-1 antibody binding, respectively). Panel B is a graph of the concentration of cRGDFG (SEQ ID NO: 12; where F is a D amino acid) versus adhesion of αIIbβ3 CHO K1 cells. The peptide cRGDFG (SEQ ID NO: 12), a cyclic peptide that specifically binds to α5 and αV integrins, inhibits CHO K1 cell adhesion to 0.5% GRGDSC (SEQ ID NO: 11) SAMs. Panels C-F are photomicrographs of cells adhering to GRGDSC (SEQ ID NO: 11). Panel C shows that CHO K1 cells adhere to 0.5% GRGDSC (SEQ ID NO: 11) SAMs in the absence of cRGDFG (SEQ ID NO: 12), but fail to adhere to the same substrate in the presence of [cRGDFG (SEQ ID NO: 12)]=300 μM (panel D). Panel E shows that αIIbβ3 CHO K1 cells adhere to 0.5% GRGDSC (SEQ ID NO: 11) SAMs in the presence of [cRGDFG (SEQ ID NO: 12)]=300 μM, but do not bind in the presence of PAC-1 antibody).

FIG. 3 depicts photomicrographs of αIIbβ3 CHO cell adhesion and cytoskeletal structure on model substrates. Optical micrographs of αIIbβ3 CHO cells incubated with [cRGDFG (SEQ ID NO: 12)]=300 μM that adhered and spread on 0.5% SAMs presenting GRGDSC (SEQ ID NO: 11) (A), HHLGGAKQAGDVC (SEQ ID NO: 21) (B) and a mixed monolayer comprised of both peptides (C). As a control, αIIbβ3 CHO cells adhered and spread on a dodecanethiol monolayer with adsorbed Fbg (D). Scale bars are 300 μm. Immunofluorescence staining of αIIbβ3 CHO cells incubated with [cRGDFG (SEQ ID NO: 12)]=300 μM that adhered and spread on 0.5% SAMs presenting GRGDSC (SEQ ID NO: 11) (E), HHLGGAKQAGDVC (SEQ ID NO: 21) (F), a mixed monolayer comprised of both peptides (G) and the Fbg control (H). Focal adhesions were visualized with an anti-vinculin antibody (red), actin stress fibers were visualized with AF 488 phalloidin (green), and nuclei were visualized with DAPI (4′,6-diamidino-2-phenylindole) (blue). Scale bars are 50 μm.

FIG. 4 depicts photomicrographs of αIIbβ3 CHO K1 cell binding in presence or absence of specified peptides. (Panel A) Truncation of HHLGGAKQAGDV (SEQ ID NO: 1) ligand to find minimal binding sequence using peptides HHLGGAC (SEQ ID NO: 13), KQAGDVC (SEQ ID NO: 14), and GGAKQAC (SEQ ID NO: 15). Optical micrographs of αIIbα3 CHO K1 cells incubated with [cRGDFG(SEQ ID NO: 12)]=300 μM that adhered and spread on 0.5% KQAGDVC (panel D)(SEQ ID NO: 14) but failed to adhere to 0.5% HHLGGAC (panel B)(SEQ ID NO: 13) and 0.5% GGAKQAC (panel C)(SEQ ID NO: 15) SAMs. When KQAGDVC (SEQ ID NO: 14) was truncated from the N terminus, αIIbβ3 CHO K1 cells adhered to 0.5% QAGDVC (panel E)(SEQ ID NO: 16; E) and 0.5% AGDVC (panel F)(SEQ ID NO: 17) SAMs, but failed to adhere to 0.5% GDVC (panel G)(SEQ ID NO: 18) SAMs. When KQAGDVC (SEQ ID NO: 14) was truncated from the C terminus, αIIbβ3 CHO K1 cells adhered to 0.5% KQAGDC (panel H)(SEQ ID NO: 19) SAMs, but failed to adhere to 0.5% KQAGC (panel I)(SEQ ID NO: 20) SAMs. Scale bars are 300 μm.

FIG. 5 depicts graphs of the percent of cell adhesion versus negative log of the concentration of the peptide ligand. Inhibition of αIIbβ3 CHO K1 cells incubated with [cRGDFG (SEQ ID NO: 12)]=300 μM to monolayers presenting 0.5% GRGDSC (panel A)(SEQ ID NO: 11; A), 0.5% HHLGGAKQAGDVC (panel B)(SEQ ID NO: 21) and 0.5% AGDVC (panel C)(SEQ ID NO: 17). Suspended cells were treated with either soluble GRGDS (SEQ ID NO: 4; solid diamonds), soluble HHLGGAKQAGDV (SEQ ID NO: 1; solid ovals), or soluble AGDV (SEQ ID NO: 22; solid triangles) at concentrations from 1 pM to 100 μM and then allowed to adhere to the model substrates. Cell adhesion was quantized as normalized units relative to adhesion in the absence of soluble peptides. The scrambled peptides GGRDGS (SEQ ID NO: 23; open diamonds), GHHLGGADQAGKV (SEQ ID NO: 24; open ovals) and GADGV (SEQ ID NO: 25; open triangles) were used to examine selective inhibition. Error bars are omitted for clarity.

FIG. 6 depicts the percent cell adhesion mediated by Ac-GXGDSC (SEQ ID NO: 26; Panel A) and Immunostained cells (Panel B). (A) αIIbβ3 CHO K1 cells, CHO K1 cells, BHK 21 cells and HT 1080 cells were allowed to adhere to arrays of 0.5% GXGDSC (SEQ ID NO: 27). Cell adhesion was quantized as normalized units relative to adhesion in the 0.5% Ac-GRGDSC (SEQ ID NO: 28) spot. Cells did not adhere to the spot presenting 0.5% GGRDGSC (SEQ ID NO: 70). Error bars are omitted for clarity. Immunostaining of peptides that mediated αIIbβ3 CHO K1 cell adhesion on the array. (B) 0.5% Ac-GRGDSC (SEQ ID NO: 28), (C) 0.5% Ac-GGGDSC (SEQ ID NO: 29), (D) 0.5% Ac-GAGDSC (SEQ ID NO: 30), (E) 0.5% Ac-GKGDSC (SEQ ID NO: 31), (F) 0.5% Ac-GVGDSC (SEQ ID NO: 32), (G) 0.5% Ac-GIGDSC (SEQ ID NO: 33) and (H) 0.5% Ac-GLGDSC (SEQ ID NO: 34). Focal adhesions were visualized with an anti-vinculin antibody (red), actin stress fibers were visualized with AF 488 phalloidin (green), and nuclei were visualized with Hoescht (blue).Scale bars are 50 μm. (I) 5X4 SAM array used for this work.

### DETAILED DESCRIPTION

In one aspect, the present invention relates to compounds of formula I, II, III, IV, and V. In certain embodiments, these compounds are useful as platelet aggregation inhibitors (PAIs). Compounds of the present invention (e.g., compounds of Formulas I-V) can be prepared by applying synthetic methodologies known in the art. For example, compounds may be synthesized using solid-phase peptide synthesis methodologies. Cleavage of the peptide from the solid-phase typically yields linear peptides. These linear peptides may be cyclized to form a compound of formula II-V. For example, the linear peptides (e.g., between X¹ and X⁵ for compounds of formula II) may be cyclized under conditions that provide for a bond between a residue that is N-terminal to the G* and a residue that that is C-terminal to the G* to generate a compound of formulas II-V.

A “residue capable of forming a bond between X¹ and X⁵ to obtain a cyclic compound” may be an amino acid or non-amino acid that may be reacted to form a bond between X¹ and X⁵. For example, X¹ or X⁵ may be a naturally occurring amino acid (i.e., Ala, Cys, Asp, Glu, Phe, Gly, His, Ile, Lys, Leu, Met, Asn, Pro, Gln, Arg, Ser, Thr, Val, or Tyr) or a residue having a sulfhydryl group such as Mpr (mercaptopropionyl), Mvl (mercaptovaleryl), Cys, Pen (Penicillamine), Pmp (β₁,β-pentamethylene-β-mercaptopropionic acid), and Pmc (amino-β₁,β-pentamethylene-β-mercaptopropionic acid). In certain embodiments, X¹ is not Q, K, or R.

X³ is a hydrophobic amino acid, which includes naturally occurring and non-naturally occurring hydrophobic amino acids. Examples of naturally occurring hydrophobic amino acids include Ala, Val, Ile, Gly, and Leu. Examples of non-naturally occurring hydrophobic amino acids include those in the following table:

| Amino acid | Structure |
|---|---|
| Fmoc-Abu-OH | 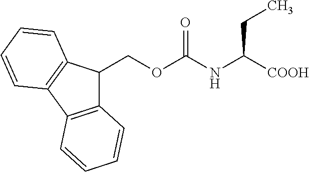 |
| Fmoc-Aib-OH | 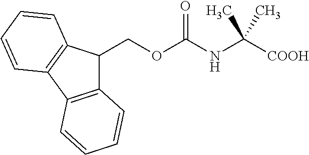 |
| Fthoc-α-t.-butylglycine | 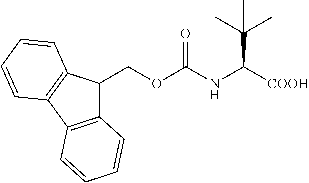 |
| Fmoc-Cha-OH | 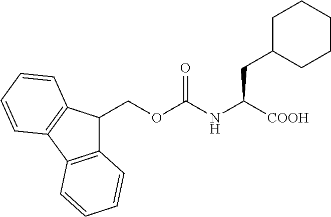 |
| Fmoc-Lys(Dnp)-OH | 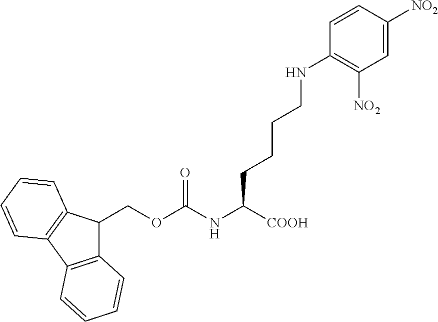 |
| Fmoc-Met(O)-OH | 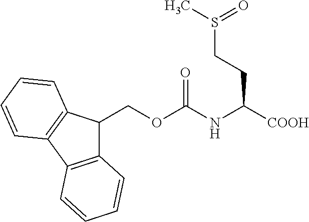 |
| Fmoc-Met(O₂)-OH | 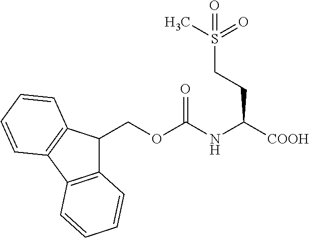 |
| Fmoc-Nle-OH | 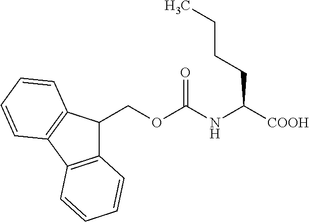 |
| Fmoc-Nva-OH | 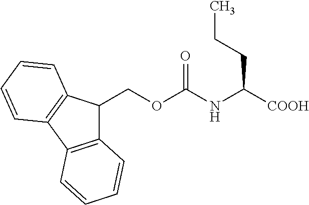 |
| Fmoc-Phe(4-Cl)-OH | 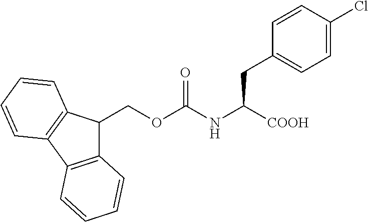 |
| Fmoc-Phe(4-F)-OH | 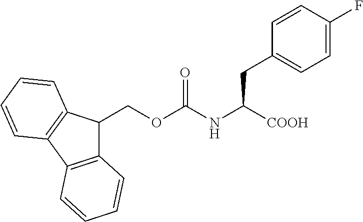 |
| Fmoc-Phe(4-NO₂)-OH | 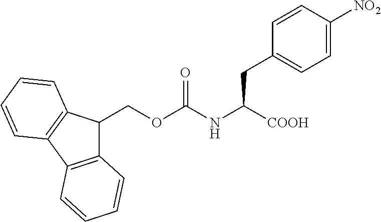 |
| Fmoc-Phg-OH | 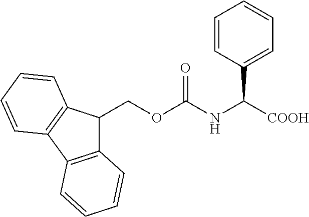 |
| Fmoc-3,4-dehydro- Pro-OH | 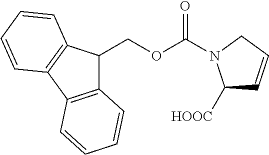 |
| Fmoc-Sta-OH | 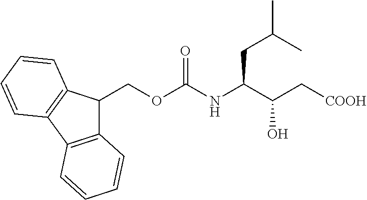 |
| Fmoc-Tic-OH | 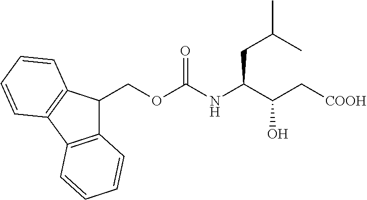 |
| Fmoc-Thi-OH | 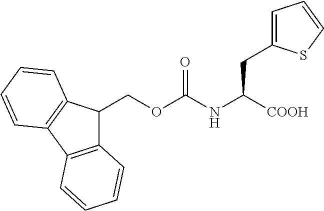 |

In certain embodiments, a linear peptide may be cyclized using a disulfide bond, between two cysteine residues or between a cysteine and a Mpr, Pen, or Mvl residue. Further examples of linkages that may be used to generate a cyclic peptide include, but are not limited to a peptide bond formed between the NH₂ of the N-terminal residue and the COOH of the carboxy terminal residue, or an amide bond (i.e., —C(O)—NH—) formed between an amino acid side chain (e.g., of a lysine) and a side chain of another amino acid (e.g., Glu or Asp), or an ester bond (i.e., —C(O)—O—) formed between a side chain hydroxyl (e.g., of a threonine) and a C-terminal COOH or COOH of a side chain of a Glu or Asp.

Cyclization may also be achieved through a click chemistry reaction (see e.g., Hein et al. (2008) Pharmaceutical Research, 25: 2216-2230). For example, X¹ may be bound to X⁵ for compounds of formula II (or X¹ may be bound to D for compounds of formula III, or X³ may be bound to X⁵ for compounds of formula IV, or X³ may be bound to D for compounds of formula V) through a 1,4-disubstituted 1,2,3-triazolyl group:

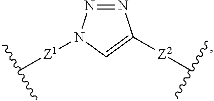

where Z¹ and Z² are independently selected —(CH₂)ₙ— groups, wherein n is an integer from 1 to 7, and wherein if n is an integer from 3 to 7, then one or two non-adjacent CH₂ groups may be replaced with groups independently selected from the group consisting of: —C(O)—, —NH—, —N(CH₃)—, —C(O)—NH—, —S—, —C(O)—O—, and —O—. In certain embodiments one CH₂ group may be replaced with groups independently selected from the group consisting of: —C(O)—, —NH—, —N(CH₃)—, —C(O)—NH—, —S—, —C(O)—O—, and —O—. This cyclization through the 1,4-disubstituted 1,2,3-triazolyl group may be achieved, for example, by the Cu¹-catalyzed Huisgen 1,3-dipolar cycloaddition of an azide and a terminal alkyne group (see e.g., Hein et al. (2008)).

Alternatively, X¹ may be bound to X⁵ through a 1,2,3-triazol-1,5-yl group (or X¹ may be bound to D for compounds of formula III, or X³ may be bound to X⁵ for compounds of formula IV, or X³ may be bound to D for compounds of formula V):

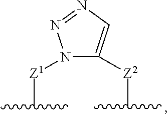

where Z¹ and Z² are independently selected —(CH₂)ₙ— groups, wherein n is an integer from 1 to 7, and wherein if n is an integer from 3 to 7, then one or two non-adjacent CH₂ groups may be replaced with groups independently selected from the group consisting of: —C(O)—, —NH—, —N(CH₃)—, —C(O)—NH—, —S—, —C(O)—O—, and —O—. In certain embodiments one CH₂ group may be replaced with groups independently selected from the group consisting of: —C(O)—, —NH—, —N(CH₃)—, —C(O)—NH—, —S—, —C(O)—O—, and —O—. This cyclization through the 1,2,3,-triazol-1,4-yl group may be achieved, for example, by the Cu¹ -catalyzed Huisgen 1,3-dipolar cycloaddition of an azide and a terminal alkyne group (see e.g., Hein et al. (2008)).

An example of a linker connecting an X¹ to an X⁵ with a 1,2,3-triazol-1,5- yl group is

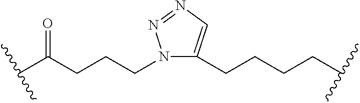

### (e.g., Z¹ is —C(O)—(CH₂)₃— and Z² is —(CH₂)₃—).

The variable X² may be absent or an amino acid sequence of independently selected amino acids that is from one to six amino acids in length. In certain embodiments, X² may be Gly, Trp, Glu, Arg, His, or Ser.

The variable X⁴ may be absent or is a sequence of independently selected amino acids that is from one to six amino acids in length. For example X⁴ may be Trp or Trp-Pro.

The variable Y¹ maybe absent, H, acetyl, or an amino protecting group. Those of skill in the art will recognize that a wide variety of protecting groups can be used as a suitable protecting group for an amino group (see e.g., Greene and Wuts, Protective Groups in Organic Synthesis, Wiley-Interscience; 3rd edition (1999)). Examples of suitable amino protecting groups include, but are not limited to, tert-butyloxycarbonyl (BOC), 9-fluorenylmethyloxycarbonyl (FMOC) group, carbobenzyloxy (Cbz), p-methoxybenzyl carbonyl (Moz), p-methoxyphenyl (PMP), tosyl (Ts), benzyl (Bn), p-methoxybenzyl (PMB), and 3,4-dimethoxybenzyl (DMPM).

The variable Y² may be —OH, NH₂, or a carboxyl protecting group. Those of skill in the art will recognize that a wide variety of protecting groups can be used as a suitable protecting group for a carboxyl group (see e.g., Greene and Wuts, Protective Groups in Organic Synthesis, Wiley-Interscience; 3rd edition (1999)). Examples of suitable carboxyl protecting groups include, but are not limited to, substituted methyl groups, phenyl, tetrahydropyranyl, tetrahydropyranyl, cyclopentyl, cyclohexyl, 3-buten-1-yl, and —SiR¹⁴R ¹⁵R¹⁶, where R¹⁴, R¹⁵, and R¹⁶ are each independently selected from the group consisting of: a (C₁-C₆)alkyl, and a phenyl.

Examples of suitable carboxy protecting groups include 9-fluorenylmethyl, methoxymethyl, methylthiomethyl, methoxyethoxymethyl, 2-(trimethylsilyl)ethoxy-methyl, benzyloxymethyl, phenacyl, p-bromophenacyl, a methylphenacyl, p-methoxyphenacyl, carboxamidomethyl, N-phthalimidomethyl, 2,2,2-trichloroethyl, 2-haloethyl, co-chloroalkyl, 2-(trimethylsily)ethyl, 2-methylthioethyl, 1,3-dithianyl-2-methyl, 2(p-nitrophenylsulfenyl)-ethyl, 2-(p-toluenesulfonyl)ethyl, 2-(2′-pyridyl)ethyl, 2-(diphenylphosphino)ethyl, 1-methyl-l-phenylethyl, allyl, 4-(trimethylsily)-2-buten-1-yl, cinnamyl, a-methylcinnamyl, p-(methylmercapto)-phenyl, benzyl, triphenylmethyl, diphenylmethyl, bis(o-nitrophenyl)methyl, 9-anthrylmethyl, 2-(9,10-dioxo)anthrylmethyl, 5-dibenzo-suberyl, 1-pyrenylmethyl,2-(trifluoromethyl)-6-chromylmethyl, 2,4,6-trimethylbenzyl, p-bromobenzyl, o-nitrobenzyl, p-nitrobenzyl, p-methoxybenzyl, 2,6-dimethoxybenzyl, 4-(methylsulfinyl)benzyl, 4-sulfobenzyl, and piperonyl.

Examples of compounds of formula II include, but are not limited to compounds of the following formula, where X³ is A, V, L, I, or G:

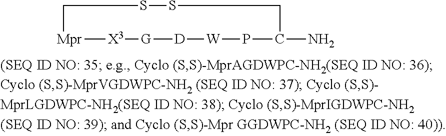

Additional examples of compounds of formula II include, but are not limited to compounds of the following formula, where X³ is A, V, L, I, or G:

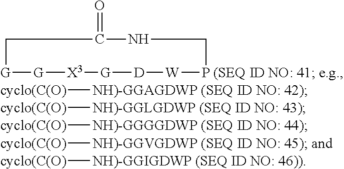

Further examples of compounds of formula II also include, but are not limited to compounds of the following formula, where X³ is A, V, L, I, or G:

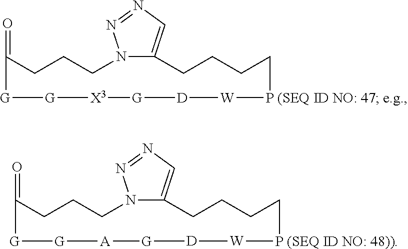

Yet other examples of compounds of formula II include, but are not limited to compounds of the following formula, where X⁴ is W, E, R, H, G, or S:

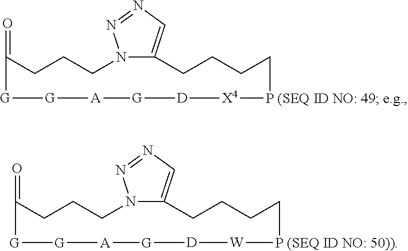

The cyclization may also be achieved instead by a direct amide bond between the N-terminal Gly and the C-terminal Pro:

Examples of compounds of formula II also include, but are not limited to compounds of the following formula, where X² is W, E, R, H, G, or S:

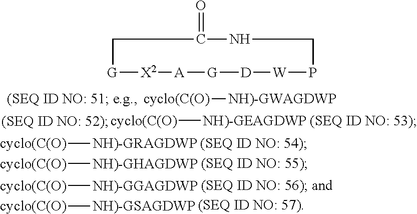

Examples of compounds of formula II also include, but are not limited to cyclo(C(O)—NH)-GGAGDWP (SEQ ID NO: 58), cyclo(C(O)—NH)-GAGDWP (SEQ ID NO: 59), cyclo(C(O)—NH)-GGAGDW (SEQ ID NO: 60), and cyclo(C(O)—NH)-GAGDW (SEQ ID NO: 61). The Ala residues of those compounds may be replaced with Val, Ile, Leu or Gly. The N-terminal and C-terminal residues may also be replaced with a 1,2,3-triazol-1,5-yl group, such as

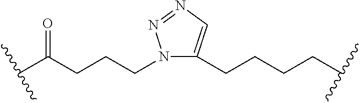

Compounds of formula III include, but are not limited to cyclo(C(O)—NH)-GGAGD (SEQ ID NO: 62), cyclo(C(O)—NH)-GGVGD (SEQ ID NO: 63), cyclo(C(O)—NH)-GGIGD (SEQ ID NO: 64), cyclo(C(O)—NH)-GGLGD (SEQ ID NO: 65), and cyclo(C(O)—NH)-GGGGD (SEQ ID NO: 66).

Compounds of formula IV include, but are not limited to cyclo(C(O)—NH)-AGDWP (SEQ ID NO: 67) and cyclo(C(O)—NH)-AGDW (SEQ ID NO: 68). Compounds of formula V include cyclo(C(O)—NH)-AGD (SEQ ID NO: 69), however the Ala may also be Val, Ile, Leu, or Gly.

Typically, the linkages between any of X¹ to X², X² to X³, X³ to G*, G* to D, D to X⁴, X⁴ to X⁵, or between any amino acid residues of X⁴ or X² are peptide bonds. In certain embodiments, one or more of those one or more peptide linkages may optionally be replaced by one or more linkages independently selected from the group consisting of: —CH₂NH—, —CH₂S—, CH₂CH₂—, —CH═CH— (cis and trans), —COCH₂—, —CH(OH)CH₂— and —CH₂SO—.

Evaluation of Compounds

Compounds of Formulas I-V can be assayed for their abilities to inhibit binding of αIIbβ3 CHO K1 cells to a SAM of a HHLGGAKQAGDVC (SEQ ID NO: 21) peptide. Compounds of the present invention may also be assayed in vitro for their ability to inhibit the aggregation of platelets. Compounds of Formulas I-V may be also evaluated using the methods set out in Gustafsson et al. (2001) Thrombosis Research 101: 171-181; Coller et al. (1991) Ann NYAcad Sci 614:193-213; Hanson et al. (1985) Arteriosclerosis 5: 595-603; Falati et al. (2004) Methods in Molecular Biology 272: 187-197; and Sakariassen et al. (2001) Thrombosis Research 104:149-174.

Pharmaceutically Acceptable Salts and Solvents

The compounds to be used in the present invention can exist in unsolvated forms as well as solvated forms, including hydrated forms. In general, the solvated forms, including hydrated forms are intended to be encompassed within the scope of the present invention.

Compounds of Formulas I-V may be capable of further forming pharmaceutically acceptable salts, including but not limited to acid addition and/or base salts. Examples of suitable pharmaceutically acceptable salts can be found for example in Stahl and Wermuth, Handbook of Pharmaceutical Salts: Properties, Selection, and Use, Wiley-VCH, Weinheim, Germany (2002); and Berge et al., “Pharmaceutical Salts,” J. of Pharmaceutical Science, 1977; 66:1-19.

Pharmaceutically acceptable acid addition salts of the compounds of Formulas I-V may include non-toxic salts derived from inorganic acids such as hydrochloric, nitric, phosphoric, sulfuric, hydrobromic, hydriodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include the acetate, aspartate, benzoate, besylate (benzenesulfonate), bicarbonate/carbonate, bisulfate, caprylate, camsylate (camphor sulfonate), chlorobenzoate, citrate, edisylate (1,2-ethane disulfonate), dihydrogenphosphate, dinitrobenzoate, esylate (ethane sulfonate), fumarate, gluceptate, gluconate, glucuronate, hibenzate, hydrochloride/chloride, hydrobromide/bromide, hydroiodide/iodide, isobutyrate, monohydrogen phosphate, isethionate, D-lactate, L-lactate, malate, maleate, malonate, mandelate, mesylate (methanesulfonate), metaphosphate, methylbenzoate, methylsulfate, 2-napsylate (2-naphthalene sulfonate), nicotinate, nitrate, orotate, oxalate, palmoate, phenylacetate, phosphate, phthalate, propionate, pyrophosphate, pyrosulfate, saccharate, sebacate, stearate, suberate, succinate sulfate, sulfite, D-tartrate, L-tartrate, tosylate (toluene sulfonate), and xinafoate salts, and the like of compounds of Formula I-V. Also contemplated are the salts of amino acids such as arginate, gluconate, galacturonate, and the like.

Acid addition salts of the basic compounds may be prepared by contacting the free base form with a sufficient amount of the desired acid to produce the salt. The free base form may be regenerated by contacting the salt form with a base and isolating the free base.

Pharmaceutically acceptable base addition salts may be formed with metals or amines, such as alkali and alkaline earth metal hydroxides, or of organic amines. Examples of metals used as cations are aluminum, calcium, magnesium, potassium, sodium, and the like. Examples of suitable amines include arginine, choline, chloroprocaine, N,N′-dibenzylethylenediamine, diethylamine, diethanolamine, diolamine, ethylenediamine (ethane-1,2-diamine), glycine, lysine, meglumine, N-methylglucamine, olamine, procaine (benzathine), and tromethamine.

The base addition salts of acidic compounds may be prepared by contacting a free acid form with a sufficient amount of the desired base to produce the salt. The free acid form may be regenerated by contacting the salt form with an acid and isolating the free acid.

### Pharmaceutical Compositions and Methods of Use

The present invention also provides for pharmaceutical compositions comprising a therapeutically effective amount of a compound of Formulas I-V, or a pharmaceutically acceptable salt thereof together with a pharmaceutically acceptable carrier, diluent, or excipient therefor. The phrase “pharmaceutical composition” refers to a composition suitable for administration in medical or veterinary use. The phrase “therapeutically effective amount” means an amount of a compound, or a pharmaceutically acceptable salt thereof, sufficient to inhibit, halt, or allow an improvement in the disease being treated when administered alone or in conjunction with another pharmaceutical agent or treatment in a particular subject or subject population. For example in a human or other mammal, a therapeutically effective amount can be determined experimentally in a laboratory or clinical setting, for the particular disease and subject being treated. Preferably, a compound of the present invention will cause a decrease in symptoms or a disease indicia associated with a platelet aggregation disorder as measured quantitatively or qualitatively.

It should be appreciated that determination of proper dosage forms, dosage amounts, and routes of administration is within the level of ordinary skill in the pharmaceutical and medical arts. The dose administered to a subject, in the context of the present invention should be sufficient to affect a beneficial therapeutic response in the subject over time. The term “subject” refers to a member of the class Mammalia. In certain embodiments, the “subject” is a human.

The dose will be determined by the efficacy of the particular compound employed and the condition of the subject, as well as the body weight or surface area of the subject to be treated. The size of the dose also will be determined by the existence, nature, and extent of any adverse side effects that accompany the administration of a particular compound in a particular subject. In determining the effective amount of the compound to be administered in the treatment or prophylaxis of the disease being treated, the physician can evaluate factors such as the circulating plasma levels of the compound, compound toxicities, and/or the progression of the disease, etc.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. For example, a unit dose may contain 1-50 mg of a compound of the present invention. The composition can, if desired, also contain other compatible therapeutic agents. For administration, compounds of the present invention can be administered at a rate determined by factors that can include, but are not limited to, the pharmacokinetic profile of the compound, contraindicated drugs, and the side effects of the compound at various concentrations, as applied to the mass and overall health of the subject. Administration can be accomplished via single or divided doses.

The dosage can range broadly depending upon the desired affects and the therapeutic setting. Typically, dosages will be between about 0.01 and 10 mg/kg, preferably between about 0.01 to 0.1 mg/kg body weight. In certain embodiments a dose may be between 0.5 to 20 mg.

A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Liquid form preparations include solutions, suspensions.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of the present invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride or the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized.

The compounds of the present invention and pharmaceutical compositions comprising a compound of the present invention can be administered to treat a subject suffering from a platelet aggregation disorder. A “platelet aggregation disorder” is a disorder or condition whose symptoms or disease indicia are associated with the aggregation of platelets. Examples of platelet aggregation disorders include, but are not limited to, a platelet associated ischemic disorder, platelet loss during extracorporeal circulation of blood, platelet aggregation, embolization or consumption of extracorporeal circulation, clot formation, myocardial infarction, stroke, and acute coronary syndrome.

Platelet aggregation disorders can be treated prophylactically, acutely, and chronically using compounds of the present invention, depending on the nature of the disease. The term “treatment” includes the acute, chronic, or prophylactic diminishment or alleviation of at least one symptom or characteristic associated with or caused by the disease being treated. For example, treatment can include diminishment of several symptoms of a disease, inhibition of the pathological progression of a disease, or complete eradication of a disease. The compounds of the invention are useful therapeutically to treat thrombus formation. Indications appropriate to such treatment include, without limitation, atherosclerosis and arteriosclerosis, acute myocardial infarction, chronic unstable angina, transient ischemic attacks and strokes, peripheral vascular disease, arterial thrombosis, preeclampsia, embolism, restenosis and/or thrombosis following angioplasty, carotid endarterectomy, anastomosis of vascular grafts, and chronic cardiovascular devices (e.g., in-dwelling catheters or shunts “extracorporeal circulating devices”). These syndromes represent a variety of stenotic and occlusive vascular disorders thought to be initiated by platelet activation on vessel walls.

The compounds may be used for treatment or abortion of arterial thrombus formation, in unstable angina and arterial emboli or thrombosis, as well as treatment of myocardial infarction (MI) and mural thrombus formation post-MI. For brain-related disorders, treatment of transient ischemic attack and treatment of thrombotic stroke or stroke-in-evolution are included.

The compounds may also be used to treat platelet aggregation, embolization, or consumption in extracorporeal circulations, including improving renal dialysis, cardiopulmonary bypasses, hemoperfusions, and plasmapheresis.

The compounds may be used to treat platelet aggregation, embolization, or consumption associated with intravascular devices, and administration results in improved utility of intraaortic balloon pumps, ventricular assist devices, and arterial catheters.

The compounds may be used in treatment of venous thrombosis as in deep venous thrombosis, thrombosis of the inferior vena cava (IVC), renal vein or portal vein thrombosis, and pulmonary venous thrombosis.

Various disorders involving platelet consumption, such as thrombotic thrombocytopenic purpura are also treatable.

In therapeutic applications, the compounds of the present invention can be prepared and administered in a wide variety of oral and parenteral dosage forms. The term “administering” refers to the method of contacting a compound with a subject. Thus, the compounds of the present invention can be administered by injection, that is, intravenously, intramuscularly, intracutaneously, subcutaneously, intraduodenally, parentally, or intraperitoneally. In certain embodiments, the compounds of Formulas I-V may be administered intravenously. Also, the compounds described herein can be administered by inhalation, for example, intranasally. Additionally, the compounds of the present invention can be administered transdermally, topically, and via implantation. In certain embodiments, the compounds of the present invention are delivered orally. The compounds can also be delivered rectally, bucally, intravaginally, ocularly, or by insufflation.

In addition, the compounds of the present invention may be used in numerous nontherapeutic applications where inhibiting platelet aggregation is desired. For example, improved platelet and whole blood storage can be obtained by adding sufficient quantities of the peptides, the amount of which will vary depending upon the length of proposed storage time, the conditions of storage, the ultimate use of the stored material, etc.

### EXAMPLES

Antibodies and Reagents

All reagents were used as received. All amino acids and peptide synthesis reagents were purchased from AnaSpec, Inc. (Fremont, Calif.). Anti-integrin antibody PAC-1 (anti-αIIbβ3) was obtained from BD Biosciences. Other anti-integrin antibodies, M-148 (anti-αIIb) and H-96 (anti-β3) were purchased from Santa Cruz Biotechnology. Monoclonal anti-vinculin antibody was obtained from Sigma. Texas Red goat anti-mouse IgG, AF 488 goat anti-mouse IgG, AF 488 goat anti-rabbit IgG and AF 488 phalloidin were obtained from Invitrogen. 4′,6-Diamidino-2-phenylindole (DAPI) was obtained from Molecular Probes. Human Fbg was obtained from Sigma. FuGENE transfection reagent was purchased from Roche. F-12K cell culture medium was purchased from ATCC. All other cell culture media and reagents were obtained from Gibco, with the exception of geneticin and zeocin, which were from Invitrogen.

Peptide Synthesis

Linear peptides were synthesized manually following standard Fmoc peptide synthesis protocols using Fmoc-Rink amide MBHA resin (AnaSpec, Inc., Fremont, Calif.). The cyclic peptide cRGDFG (SEQ ID NO: 12), where F is a D amino acid, was synthesized using standard Fmoc peptide synthesis protocol using Fmoc-Asp(WangLL)-ODmab resin. The Dmab group was cleaved using 2% hydrazine in DMF for 20 minutes, and the peptide was cyclized by treatment with 3 equivalents of HOBt (1-hydroxy-benzotriazole) and DIC (diisopropylcarbodiimide) for 4 hours. All peptides were purified by reverse phase HPLC using a C18 column (Waters) and characterized with Matrix-Assisted Laser Desorption/Ionization Time of Flight Mass Spectrometry (MALDI-TOF MS).

Preparation of Monolayers

Monolayers were prepared as described (Houseman, et al., 2003). Glass coverslips were sonicated for 30 minutes in deionized ultrafiltered (DIUF) water and then 30 minutes in ethanol and then dried under a stream of nitrogen. Titanium (4 nm) and then gold (29 nm) were evaporated onto the coverslips using an electron beam evaporator (Boc Edwards) at a rate of 0.05-0.10 nm/s and at a pressure of 1 μTorr. The gold-coated coverslips were immersed in an ethanolic solution of maleimide-terminated disulfide (1%) and tri(ethylene glycol)-terminated disulfide (99%) overnight at room temperature. The total disulfide concentration was 1 mM. The substrates were washed with DIUF water, ethanol and then dried under a stream of nitrogen.

cDNA Constructs, Transfection and Cell Culture

The pcDNA3.1 vectors that express human αIIb or β3, respectively, have been described previously (see e.g., Litvinov et al. (2006) Biochemistry, 45: 4957-4964). CHO K1, BHK 21 and HT1080 cells were purchased from ATCC. All CHO cells were cultured at 37° C. and 5% CO₂ using F12K medium supplemented with 10% FBS and pen/strep. Other cell lines were cultured in the same conditions using medium suggested by ATCC. CHO K1 cells were transfected with αIIb and β3 cDNA with FuGENE following the supplier's protocol. Transfected cells were selected with 1 mg/mL zeocin and 1 mg/mL geneticin.

Cell Adhesion Assay

Cysteine-terminated peptides were immobilized onto maleimide-presenting SAMs by adding 40 μL of 1 mM peptide solution to the biochip and incubating at 37° C. for 1 hour. Substrates were rinsed with DIUF water and ethanol and dried under a stream of nitrogen. αIIbβ3 CHO K1 cells were detached from culture plates with 1 mM EGTA/2 mM EDTA in PBS (phosphate-buffered saline), rinsed with serum-free F-12K medium, centrifuged and resuspended in serum-free F-12K medium supplemented with cRGDFG (SEQ ID NO: 12) (300 μM) and 1× penicillin/streptomycin. After 15-minute of incubation, cells (200,000/mL) were added to substrates in 24-well culture plates. After 2 hour incubation at 37° C. and 5% CO₂, media was exchanged for F-12K medium supplemented with 10% FBS, 1× penicillin/streptomycin and cRGDFG (SEQ ID NO: 12) (300 μM). After 2 hours, the substrates were washed gently with warm PBS, and adherent cells were immediately fixed with 4% formaldehyde for 30 minutes, visualized with a 20× objective of a Zeiss Axiovert 200 inverted microscope, and photographed. For cell adhesion assays mediated by Ac-GXGDSC (SEQ ID NO: 26), αIIbβ3 CHO K1 cells were incubated on a 5×4 circle (1 mm diameter) array for 1 hour with serum-free F-12K medium supplemented with cRGDFG (SEQ ID NO: 12) (300 μM) and 1× penicillin/streptomycin, followed by media exchange to F-12K medium supplemented with 10% FBS, 1× penicillin/streptomycin and cRGDFG (SEQ ID NO: 12) (300 μM. The substrates were washed with warm PBS, and adherent cells were immediately fixed and permeabilized with 4% formaldehyde and 0.5% Triton-X for 30 minutes. Fixed cells were stained with DAPI, visualized and photographed with a 20× objective as described. The number of adherent cells per field of view was counted using Image J software. Cells were counted in at least three fields for each circle, and each experiment was repeated 3 times. The degree of adhesion is reported as a percentage of cells that adhere relative to control experiments with the peptide Ac-GRGDSC (SEQ ID NO: 28).

Immunostaining

αIIbβ3 CHO K1 cells were allowed to adhere and spread on substrates as described above. Substrates were washed gently with PBS, and fixed and permeabilized as described above. Cells were stained with monoclonal anti-vinculin IgG to visualize focal adhesions, phalloidin to visualize stress fibers and DAPI to visualize the nucleus. Substrates were rinsed thoroughly with PBS and mounted on microscope slides with Aqua Poly/Mount (PolySciences Inc.). Substrates were visualized with a 100× objective of a Zeiss Axiovert 200 inverted microscope, and photographed.

Cell Adhesion Inhibition Assay

Suspensions of αIIbβ3 CHO K1 cells (100,000/mL) were incubated with cRGDFG (SEQ ID NO: 12) (300 μM) in serum-free F-12K medium with 1 mg/mL BSA (bovine serum albumin) and 1× penicillin/streptomycin for 15 minutes at 37° C. and 5% CO₂. These suspensions were then supplemented with soluble peptides at concentrations ranging from 1 pM to 100 μM and further incubated for 15 minutes at 37° C. and 5% CO₂. Cells were then added to monolayers and incubated at 37° C. and 5% CO₂ for 2 hours, then media was exchanged for F-12K medium supplemented with 10% FBS and 1× penicillin/streptomycin. The substrates were further incubated for 2 hours. The substrates were washed gently with warm PBS, and adherent cells were immediately fixed and permeabilized with 4% formaldehyde and 0.5% Triton-X for 30 minutes. Fixed cells were stained with DAPI, visualized and photographed with a 20× as described previously. The number of adherent cells per field of view was counted using Image J. Cells were counted in at least five fields for each substrate, and each experiment was repeated 3 times. The degree of inhibition is reported as a percentage of cells that adhere relative to control experiments in the absence of soluble peptide.

Results

Experimental Design

Model substrates were used that would mimic the extracellular matrix to compare the roles played by the GRGDS (SEQ ID NO: 4) and HHLGGAKQAGDV (SEQ ID NO: 1) sequences in Fbg for their ability to mediate the attachment and spreading of cells in an αIIbβ3-dependent manner. The model substrates were self-assembled monolayers that presented the peptide ligands against a tri(ethylene glycol)-terminated background (Mrksich, 2009). The use of monolayers for studies of cell adhesion are notable because they allow the identities, densities and orientations of the peptides to be controlled and at the same time are effective at preventing the non-specific adsorption of protein and therefore remodeling of the matrix. The latter benefit owes to the terminal oligo(ethylene glycol) groups on the monolayer. Several previous reports have established the suitability of these model substrates for mechanistic studies of cell adhesion (Kato et al., 2004; Feng et al., 2004; Houseman et al, 2001; Houseman et al, 2003; Murphy et al., 2004).

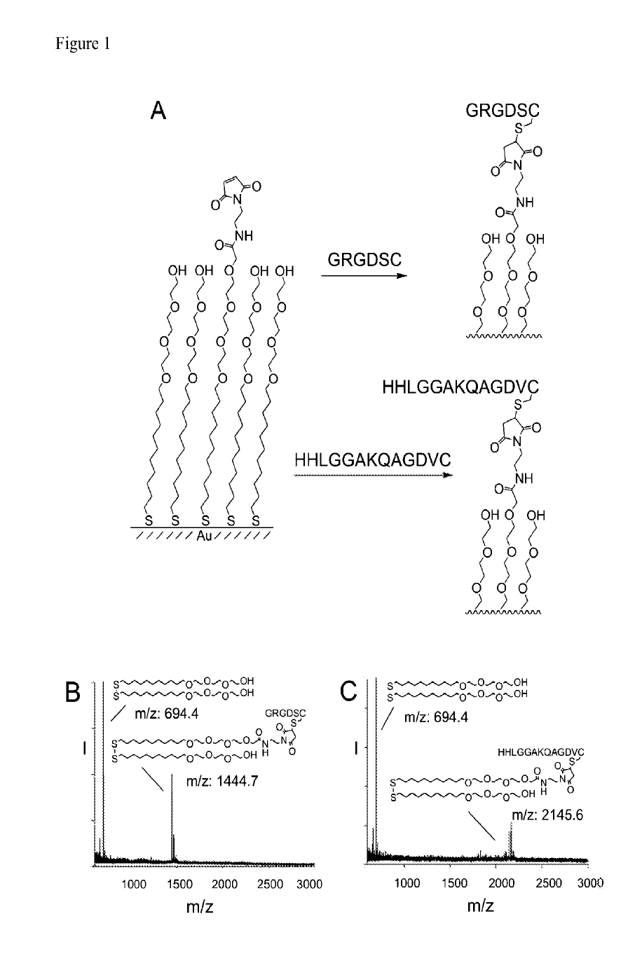

Monolayers were prepared that presented a mixture of maleimide groups and tri(ethylene glycol) groups with the former present at a density of 0.5% (relative to total alkanethiolate). The monolayers were treated with cysteine-terminated peptides to immobilize the ligands (FIG. 1A). In each case mass spectrometry was used to verify immobilization of the peptides in high yield (Su et al., 2002). For example, a mass spectrum of monolayer to which the peptide GRGDSC (SEQ ID NO: 11) was immobilized showed the expected peaks for the sodium adducts of symmetric glycol-substituted disulfide (m/z=694.4) and the mixed disulfide containing one glycol group and one GRGDSC (SEQ ID NO: 11) terminated alkanethiol (m/z: 1444.7) (FIG. 1B). Similarly, a mass spectrum of a monolayer to which the peptide HHLGGAKQAGDVC (SEQ ID NO: 21) had been immobilized displayed peaks for the sodium adduct of the symmetrical glycol disulfide (m/z: 694.4) and the mixed disulfide composed of one glycol group and one HHLGGAKQAGDVC (SEQ ID NO: 21) terminated alkanethiol (m/z: 2145.6) (FIG. 1C).

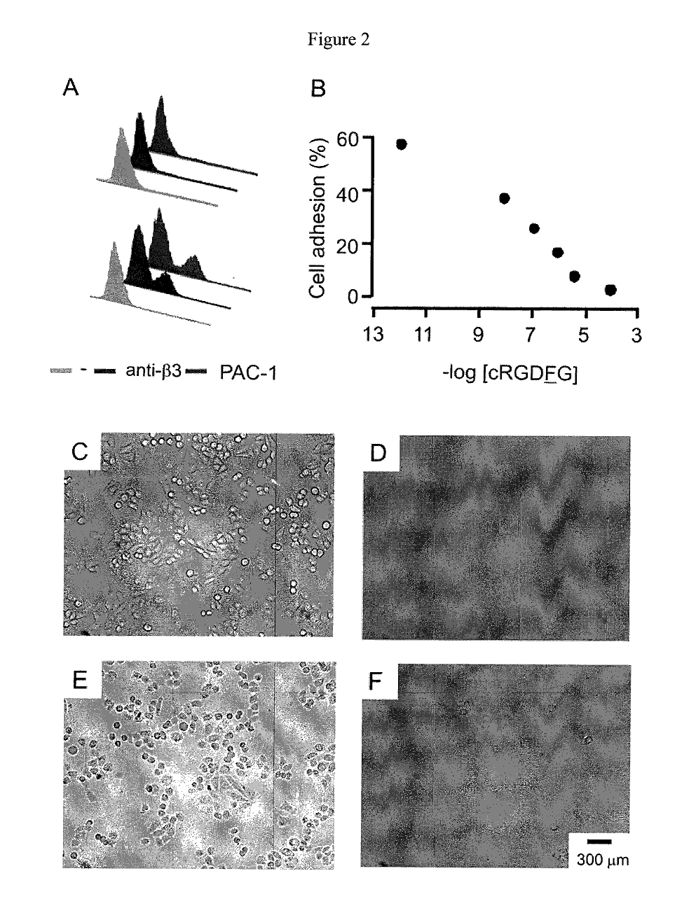

CHO K1 cells were used that were transfected with the αIIbβ3 receptor, as described previously (Salsmann et al., 2005). Cells were transfected with pcDNA3.1 vectors that coded for αIIb and β3 using FuGENE6 and analyzed by FACS after labeling with the PAC-1 (anti-αIIbβ3) and H-96 (anti-β3) antibodies. Approximately 30% of the cells expressed the receptor (FIG. 2A). Because the CHO K1 cells also express αv and α5 integrins on their surfaces, adhesion experiments were performed in the presence of the cyclic peptide cRGDFG (SEQ ID NO: 12), where F is a D amino acid. This peptide binds αvβ3 with an IC₅₀ of 1.6 nM, an affinity that is approximately 1,400-fold greater than that for the αIIbβ3 integrin (Haubner et al., 1996). It was verified that this peptide, at a concentration of 300 μM, could efficiently block the attachment of CHO K1 cells to a monolayer presenting the GRGDSC (SEQ ID NO: 11) peptide (FIG. 2D), but did not interfere with adhesion of αIIbβ3 CHO K1 cells to the same monolayer (FIG. 2E). Treatment of transfected cells with both the cyclic peptide inhibitor and the PAC-1 antibody, however, prevented cell adhesion to the monolayer (FIG. 2F). These results establish that the cRGDFG (SEQ ID NO: 12) inhibitor can be used to block av and α5 integrins on CHO cells and therefore allow studies of αIIbβ3-mediated adhesion of cells. All experiments that follow, unless stated otherwise, were performed in the presence of 300 μM cRGDFG (SEQ ID NO: 12).

Adhesion of αIIbβ3 CHO K1 Cells to GRGDSC (SEQ ID NO: 11) and HHLGGAKQAGDVC (SEQ ID NO: 21)

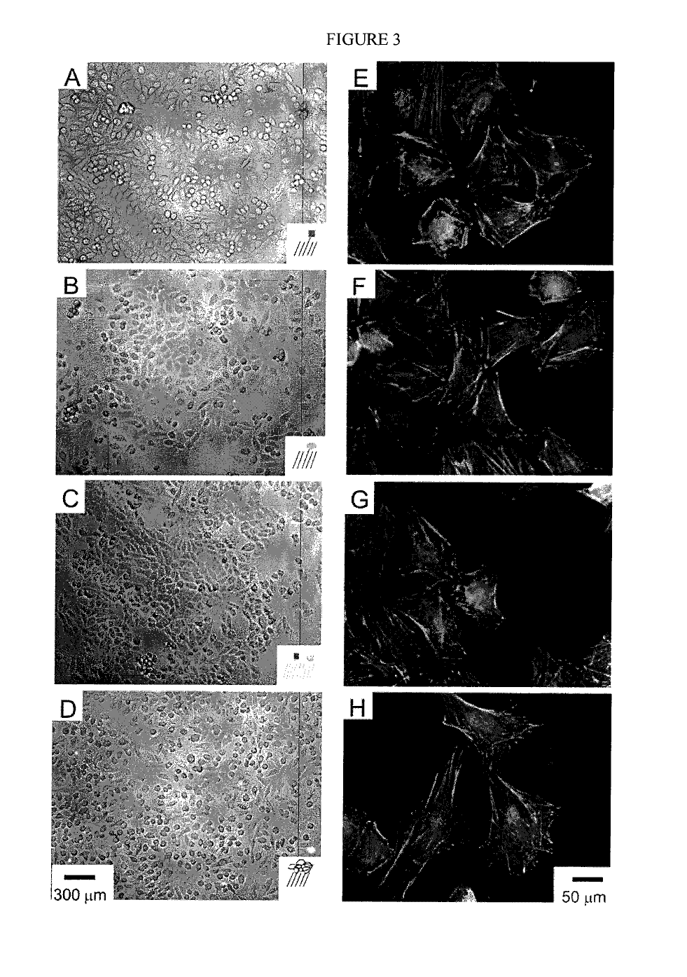

The adhesion and spreading of αIIbβ3 CHO K1 cells to monolayers presenting GRGDSC (SEQ ID NO: 11), HHLGGAKQAGDVC (SEQ ID NO: 21) and a mixture of both peptides was compared. Cells were allowed to attach to the monolayers in serum-free F-12 K medium for 2 hours after which the media was exchanged with 10% FBS F-12K medium to facilitate cell spreading. It was found that the cells adhered to monolayers presenting either the GRGDSC (SEQ ID NO: 11) or the HHLGGAKQAGDVC (SEQ ID NO: 21) peptides with comparable efficiency, in both cases assuming a flattened morphology with no refringent peripheral rim (FIG. 3A-C). The populations of cells on both substrates displayed a similar morphology, suggesting that both peptides have sufficient affinity for the receptor to support adhesion and spreading (Houseman et al., 2001). As a positive control, Fbg was adsorbed to a hydrophobic monolayer and observed a similar morphology for adherent cells (FIG. 3D). Several additional control experiments showed that the adhesion of cells on the model substrates was specific, including a lack of cell attachment to monolayers that present only glycol groups or unreacted maleimide groups (data not shown), and a lack of cell attachment to monolayers that presented the scrambled peptides GGRDGSC (SEQ ID NO: 70) or GHHLGGADQAGKVC (SEQ ID NO: 71) (data not shown).

Cytoskeletal Structure of Cells Adherent to Model Substrates

The assembly of focal adhesion and actin stress fibers of αIIbβ3 CHO K1 cells adhered to monolayers presenting GRGDSC (SEQ ID NO: 11), HHLGGAKQAGDVC (SEQ ID NO: 21) a mixture of both peptides, and adsorbed Fbg (FIGS. 3, E, F, G and H respectively) was compared. The αIIbβ3 CHO K1 cells were allowed to attach and spread on the monolayers for four hours, and then fixed the cells and probed with anti-vinculin IgG to visualize focal adhesions and with phalloidin-AF 488 to visualize actin stress fibers. Nuclei were observed with DAPI. The cells had well-developed cytoskeletal structures and mature focal adhesions along the perimeter. Cells that were allowed to adhere and spread on the three monolayers presenting the peptide ligands showed comparable cytoskeletal structures, demonstrating that the αIIbβ3-mediated adhesion to the peptide ligands is sufficient to support a well-formed cytoskeleton.

AGD is the Minimal Binding Motif for αIIbβ3

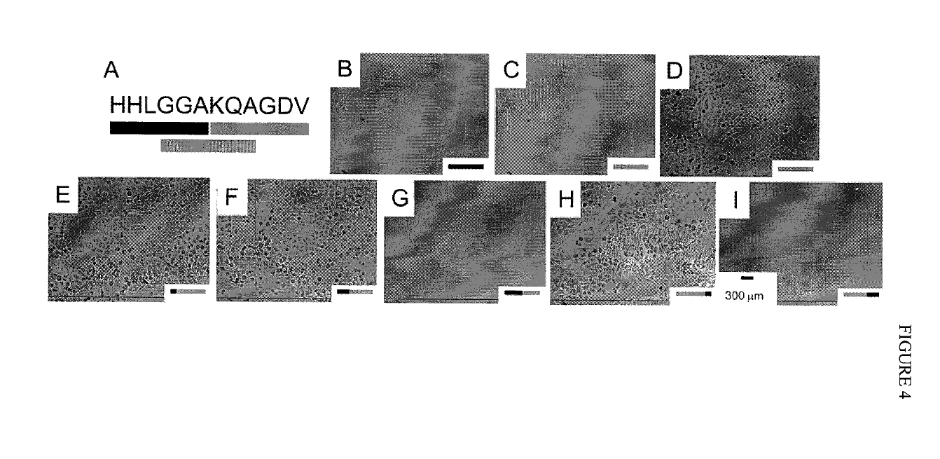

The adhesion of cells to monolayers presenting several fragments taken from the HHLGGAKQAGDV (SEQ ID NO: 1) peptide in order to define the minimal binding motif was also compared. A comparison of cell adhesion to monolayers presenting the three peptides HHLGGAC (SEQ ID NO: 13), KQAGDVC (SEQ ID NO: 14) and GGAKQAC (SEQ ID NO: 15)—corresponding to the first, last and middle six residues in the sequence—revealed that only the second sequence supported cell adhesion (FIG. 4A-D). Further studies with monolayers presenting N and C terminal truncations of this peptide revealed that the AGD tripeptide supports efficient adhesion of cells (FIG. 4, E-I).

GRGDS (SEQ ID NO: 4) and AGDV (SEQ ID NO: 22) are Competitive Ligands for αIIbβ3

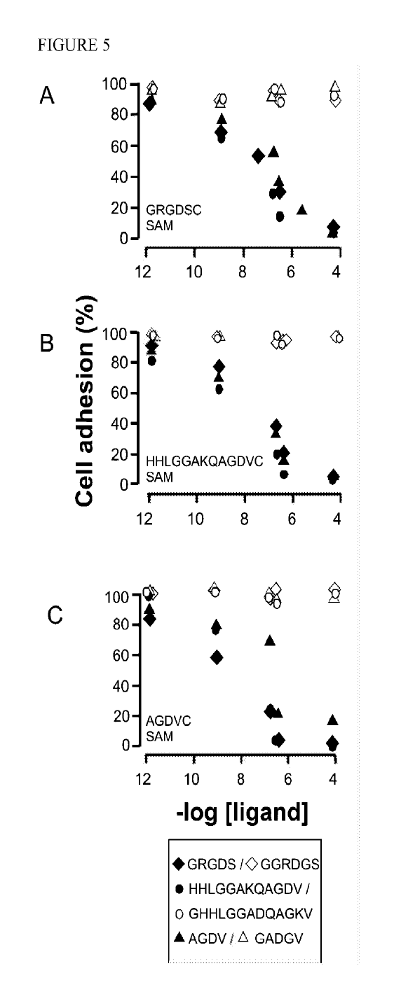

To verify that the two peptides interact with the same site on αIIbβ3, it was determined whether the peptides GRGDS (SEQ ID NO: 4), HHLGGAKQAGDV (SEQ ID NO: 1) and AGDV (SEQ ID NO: 22), in soluble form, could block the attachment of cells to the monolayers presenting these ligands. In these experiments identical suspensions of cells (100,000/mL) were first incubated in serum-free F-12K medium supplemented with cRGDFG (SEQ ID NO: 12) (300 μM) and with the soluble peptides in concentrations ranging from 1 pM to 100 μM and then applied the mixtures to monolayers presenting either the GRGDSC (SEQ ID NO: 11), HHLGGAKQAGDVC (SEQ ID NO: 21), or AGDVC (SEQ ID NO: 17) peptide (SEQ ID NO: 17) (FIG. 5). Cells were allowed to attach for 2 hours, after which media was exchanged to 10% FBS F-12K medium (supplemented with cRGDFG (SEQ ID NO: 12)) and cultured for an additional 2 hours. The slides were then rinsed and nuclei were stained with DAPI to facilitate counting of the adherent cells. It was found that each peptide could block the attachment of cells to monolayers presenting that peptide in a concentration-dependent manner. This experiment again verifies that the cell attachment is mediated by the immobilized peptide alone and not non-specific interactions between other cell-surface molecules and the monolayer. It was also found, however, that each peptide could block cell attachment to monolayers presenting any of the three peptides, demonstrating that the ligands are in fact competitive in binding αIIbβ3. The soluble peptide HHLGGAKQAGDV (SEQ ID NO: 1), for example, blocked cell attachment to a monolayer presenting GRGDS (SEQ ID NO: 4), as did the soluble AGDV peptide (SEQ ID NO: 22). Control experiments confirmed that the use of scrambled peptides GGRDGS (SEQ ID NO: 23), GHHLGGADQAGKV (SEQ ID NO: 24) and GADGV (SEQ ID NO: 25) as soluble inhibitors had no effect on cell adhesion. Again, these results show that AGDV (SEQ ID NO: 22) and GRGDS (SEQ ID NO: 4) bind to the αIIbβ3 integrin competitively and suggest a common site on the receptor.

Evaluating αIIbβ3 Specificity with a Peptide Array

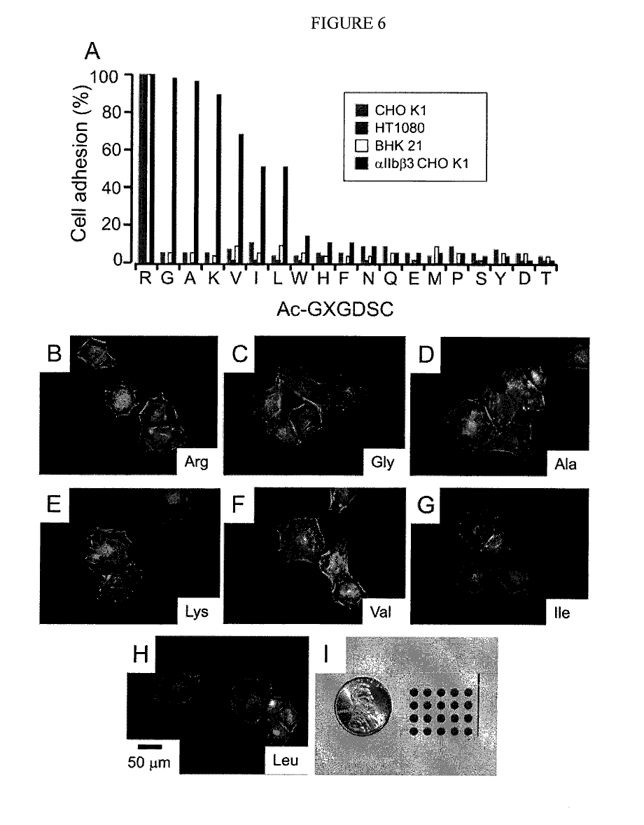

To further assay the binding of peptide ligands to the anbin integrin for XGD peptide ligands, an array was used to assess the adhesion of cells to peptides having the arginine residue substituted with each amino acid other than cysteine (e.g., A, D, E, F, G, H, I, K, L, M, N, P, Q, R, S, T, V, W, and Y). The library of Ac-GXGDSC (SEQ ID NO: 26) peptides was synthesized and immobilized in an array with each peptide occupying a 1 mm diameter region of the monolayer (FIG. 61). A suspension of αIIbβ3 CHO K1 cells was applied to the entire array and cells were allowed to attach to regions presenting the various peptides. The substrates were then rinsed and stained with DAPI, and the cells were counted to determine the relative efficiency of cell attachment to peptides in the library. It was found that the Arg residue could be substituted with the other basic residue, Lys, or with hydrophobic residues (FIG. 6, A). Substitution of the arginine residue with either glycine or alanine gave ligands that were comparable to the parent sequence and substitution with lysine and valine were only slightly less active. Substitution with leucine and isoleucine were also active, though at a lower level. Each of the other substitutions gave peptides that did not support cell adhesion.

For the five most active peptides from this array—those having Arg, Gly, Ala, Lys or Val at the first position—the αIIbβ3 CHO K1 cells had well-developed actin structures with clear vinculin-containing focal adhesions around the cell perimeter (FIG. 6, B-F). For the low affinity peptides—those containing Ile and Leu in the first position—cells displayed vinculin-containing focal adhesions around their perimeter, although these appeared less prominent than the focal adhesions supported on high affinity peptides, and the cells did not exhibit a well-defined actin cytoskeleton (FIG. 6, G-H).

This adhesion experiment was repeated with CHO-K1, BHK 21 and HT1080 cells and found that only Ac-GRGDSC (SEQ ID NO: 28) promoted significant cell adhesion, which is consistent with the known specificity of the α5 and av integrin receptors. Hence, these results show that the αIIbβ3 integrin is less specific in its recognition of peptide ligands and prefers ligands of the form XGD where X is either a basic or a hydrophobic residue.

The peptides GRGDSC (SEQ ID NO: 11) and AGDVC (SEQ ID NO: 17) Mediate αIIbβ3 CHO K1 Cell Adhesion

These results show that the platelet receptor can recognize the γ carboxy-terminal sequence by primarily interacting with the AGD motif. Further, AGD and RGD bind competitively to αIIbβ3 and have a comparable ability to mediate the adhesion of CHO cells engineered with the receptor. Indeed, it was found that αIIbβ3 CHO K1 cells adhered and spread efficiently on model substrates presenting either the GRGDSC (SEQ ID NO: 1 1) or HHLGGAKQAGDVC (SEQ ID NO: 2 1) peptides and were similar to cells that were adherent on Fbg-coated substrates. The well-developed cytoskeletal structure in cells—including the distributed focal adhesions and clear actin stress filaments—suggests that each of the peptide ligands has sufficient affinity for the platelet receptor to mediate biologically relevant adhesion. Further, control experiments establish that the adhesion of cells is mediated primarily by the immobilized ligand, since monolayers presenting no peptide or scrambled forms of the peptide failed to support cell adhesion. Inhibition experiments reveal that the two peptides bind competitively to the receptor, since either peptide in soluble form showed a dose-dependent inhibition of the attachment of cells to monolayers presenting either peptide.

Monolayers that present the fibrinogen-derived peptides HHLGGAKQAGDVC (SEQ ID NO: 21) and GRGDSC (SEQ ID NO: 1 1) were used to assay the adhesion of WIND CHO K1 cells. It was found that both peptides bind a common site on the integrin, and are both effective at mediating adhesion and spreading. A comparison of the ability of cells to adhere to a family of peptides derived from the γ ligand revealed that the AGD motif is sufficient for mediating adhesion and a peptide array revealed that the platelet integrin recognizes XGD motifs having either basic or hydrophobic residues in the first position. Hence, these results highlight the distinct ligand binding specificity of the αIIbβ3 integrin and add to the understanding of the roles of fibrinogen-derived peptides in platelet aggregation and cell adhesion.

References

Bennett, J., Shattil, S., Power, J., Gartner, T. (1988) Interaction of Fibrinogen With Its Platelet Receptor. Differential Effects of Alpha and Gamma Chain Fibrinogen Peptides of the Glycoprotein Ith-illa Complex. J. Biol. Chem., 263, 12948-12953.

Bini, A., Haidaris, P., and Kudryk, B. (2000) in Encyclopedic Reference of Vascular Biology and Pathology, A. Bikfalvi, ed. (Heidelberg, Germany: Springer), pp 107-125.

Curran, M. P., and Keating, G. M. (2005) Eptifibatide: A Review of Its use in Patients with Acute Coronary Syndromes and/or Undergoing Percutaneous Coronary Intervention. Drugs, 65, 2009-2035.

Derda, R., Li, L., Orner, B. P., Lewis, R. L., Thomson, J. A., and Kiessling, L. L. (2007) Defined Substrates for Human Embryonic Stem Cell Growth Identified from Surface Arrays. ACS Chem. Biol., 2, 347-55.

Doolittle, R., Goldbaum, D., and Doolittle, L. (1978) Designation of the Sequences Involved in the “Coiled-Coil” Interdomainal Connections in Fibrinogen: Construction of an Atomic Scale Study. J. Mol. Biol., 120, 311-325.

D'Souza, S., Ginsberg, M., Burke, T., Lam, S., and Plow, E. (1988) Localization of an Arg-Gly-Asp Recognition Site Within an Integrin Adhesion Receptor. Science, 242, 91-93.

D'Souza, S., Ginsberg, M., Burke, T., and Plow, E. (1990) The Ligand Binding Site of the Platelet Integrin Receptor GPIIb-IIIA is Proximal to the Second Calcium Binding Domain of Its α Subunit. J. Biol. Chem., 265, 3440-3446.

Farrell, D., Thiagarajan, P., Chung, D., and Davie, E. (1992) Role of Fibrinogen Alpha and Gamma Chain Sites in Platelet Aggregation. Proc. Natl. Acad. Sci. U.S.A., 89, 10729-10732.

Feng, Y., and Mrksich, M. (2004) The Synergy Peptide PHSRN and the Adhesion Peptide RGD Mediate Cell Adhesion through a Common Mechanism. Biochemistry, 43, 15811-15821.

Ginsberg, M. H., Loftus, J. C., and Plow, E. F. (1990) Ligand Binding to Integrins: Common and Ligand-Specific Recognition Mechanisms. Cell Differ. Dev., 32, 203-213.

Hantgan, R. R., Stahle, M. C., Connor, J. H., Horita, D. A., Rocco, M., McLane, M. A., Yakovlev, S. and Medved, L. (2006) Integrin αIIbβ3: Ligand Interactions are Linked to Binding-Site Remodeling. Protein Sci., 15, 1893-1906.

Haubner, R., Gratias, R., Diefenbach, B., Goodman, S., Jonczyk, A., and Kessler, H. (1996) Structural and Functional Aspects of RGD-Containing Cyclic Pentapeptides as Highly Potent and Selective Integrin αVβ3 Antagonists. J. Am. Chem. Soc., 118, 7461-7472.

Hawiger, J., Timmons, S., Kloczewiak, M., Strong, D., and Doolittle, R. (1982) γ and α Chains of Human Fibrinogen Possess Sites Reactive With Human Platelet Receptors. Proc. Natl. Acad. Sci. U.S.A., 79, 2068-2071.

Holmback, K., Danton, M. J. S., Suh, T. T., Daugherty, C. C., and Degen, J. L. (1996) Impaired Platelet Aggregation and Sustained Bleeding in Mice Lacking the Fibrinogen Motif Bound by Integrin αIIbβ3. Embo J., 15, 5760-5771.

Houseman, B. T., and Mrksich, M. (2001) The Microenvironment of Immobilized Arg-Gly-Asp Peptides is an Important Determinant of Cell Adhesion. Biomaterials, 22, 943-955.

Houseman, B. T., Gawalt, E. S. and Mrksich, M. (2003) Maleimide-Functionalized Self-Assembled Monolayers for the Preparation of Peptide and Carbohydrate Biochips. Langmuir, 19, 1522-1531.

Hu, D., White, C., Panzer-Knodle, S., Page, J. Nicholson, N., and Smith, J. (1999) A New Model of Dual Interacting Ligand Binding Sites on Integrin αIIbβ3. J. Biol. Chem., 274, 4633-4639.

Kato, M., and Mrksich, M. (2004) Using Model Substrates To Study the Dependence of Focal Adhesion Formation on the Affinity of Integrin-Ligand Complexes. Biochemistry, 43, 2699 - 2707.

Kloczewiak, M., Timmons, S., Lukas, T., and Hawiger, J. (1984) Platelet Receptor Recognition Site on Human Fibrinogen. Synthesis and Structure-Function Relationship of Peptides Corresponding to the Carboxy-Terminal Segment of the Gamma Chain. Biochemistry, 23, 1767-1774.

Lam, S. C., Plow, E., Smith, M., Andrfux, A., Ryckwaert, J., Margerie, G. and Ginsberg, M. (1987) Evidence that Arginyl-Glycyl-Aspartate Peptides and Fibrinogen γ Chain Peptides Share a Common Binding Site on Platelets. J. Biol. Chem., 262, 947-950.

Lele, M., Sajid, M., Wajih, N., and Stouffer, G. A. (2001) Eptifibatide and 7E3, but not Triptofiban, Inhibit αVβ3 Integrin-Mediated Binding of Smooth Muscle Cells to Thrombospondin and Prothrombin. Circulation, 104, 582-587.

Li, R., Hoess, R., Bennett, J., and DeGrado, W. (2003) Use of Phage Display to Probe the Evolution of Binding Specificity and Affinity in Integrins. Protein Engineering, 16, 65-72.

Mrksich, M. (2009) Using Self-Assembled Monolayers to Mimic the Extracellular Matrix. Acta Biomaterialia, 5, 832-841.

Murphy, W. L., Mercurius, K. 0., and Mrksich, M. (2004) Substrates for Cell Adhesion Prepared via Active Site-directed Immobilization of a Protein Domain. Langmuir, 20, 1026-1030.

Oshiwaka, K., and Terada, S. (1999) Ussuristatin 2, a Novel KGD-Bearing Disintegrin from Agkistrodon ussuriensis Venom. J. Biochem., 125, 31-35.

Plow, E. F., Pierscbacher, M. D., Rouslahti, E., Marguerie, G. A. and Ginsberg, M. H. (1985) The Effect of Arg-Gly-Asp Containing Peptides on Fibrinogen and von Willebrand Factor Binding to Platelets. Proc. Natl. Acad. Sci. U.S.A., 82, 8057-8061.

Salsmann, A., Schaffner-Reckinger, E., Kabile, F., Plancon, S. and Kieffer, N. (2005). A New Functional Role of the Fibrinogen RGD Motif as the Molecular Switch That Selectively Triggers Integrin αIIbβ3-Dependent RhoA Activation During Cell Spreading. J. Biol. Chem., 280, 33610-33619.

Scarborough, R. M. (1999). Development of Eptifibatide. Am. Heart. J., 138, 1093-1104.

Scarborough, R. M., Rose, J. W., Hsu, M. A., Phillips, D. R., Fried, V.A., Campbell, A. M., Nannizzi, L., and Cahro, I. F. (1991). Barbourin A. GPIIb-IIIa-Specific Specific Integrin Antagonist from the Venom of Sistrurus m. barbouri. J. Biol. Chem., 266, 9359-9362.

Scarborough, R. M., Naughton, M. N., Teng, W., Rose, J. W., Phillips, D. R., Nannizzi, L., Arfsten, A., Campbell, A. M. and Cahro, I. F. (1993). Design of Potent and Specific Integrin Antagonists. Peptide Antagonists with High Specificity for Glycoprotein IIB-IIIa. J. Biol. Chem., 268, 1066-1073.

Springer, T., Zhu, J., and Xiao, T. (2008) Structural Basis for Distinctive Recognition of Fibrinogen yC Peptide by the Platelet Integrin αIIbβ3. J. Cell Biol., 182, 791-800.

Su, J., and Mrksich, M. (2002) Using Mass Spectrometry to Characterize Self-Assembled Monolayers Presenting Peptides, Proteins and, Carbohydrates. Angew. Chem. Int. Ed., 41, 4715-4718.

Ugarova, T., Budzynski, A., Shattil, S., Ruggeri, Z., Gingsberg, M., and Plow, E. (1993) Conformational Changes in Fibrinogen Elicited by Its Interaction With Platelet Membrane Glycoprotein GPIIb-IIIA. J. Cell. Biol., 182, 791-800.

What is claimed is:

1. A compound of formula I, II, III, IV, or V: 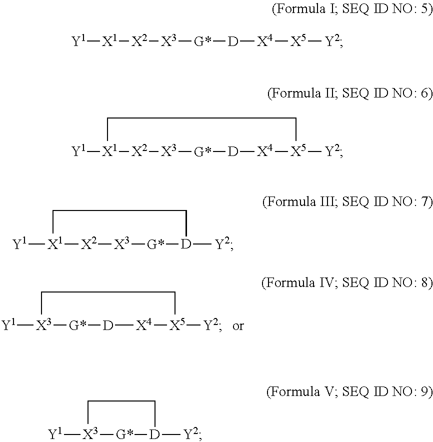
wherein [symbol]
represents a bond;
X¹ and X⁵ are independently selected residues capable of forming a bond between X¹ and X⁵ to obtain a cyclic compound;
X² is absent or is a sequence of independently selected amino acids that is from one to six amino acids in length;
X⁴ is absent or is a sequence of independently selected amino acids that is from one to six amino acids in length;
wherein the sum of the length of amino acids in X² plus X⁴ is from zero to 6 amino acids;
X³ is a hydrophobic amino acid residue; and
G* is glycyl or sarcosyl;
Y¹ is absent, H, acetyl, or an amino protecting group;
Y² is —OH, NH₂, or a carboxyl protecting group;
wherein one or more peptide linkages of Formulas I-V may optionally be replaced by a linkage selected from the group consisting of —CH₂NH—, —CH₂S—, CH₂CH₂—, —CH═CH— (cis and trans), —COCH₂—, —CH(OH)CH₂— and —CH₂SO—;
or a pharmaceutically acceptable salt thereof;
with the proviso that the following peptides are excluded from the scope of formula (I):
(SEQ ID NO: 1)
HHLGGAKQAGDV,
(SEQ ID NO: 3)
LGGAKQAGDV,
and
(SEQ ID NO: 10)
LQAGDV.

2. The compound of claim 1, or a pharmaceutically acceptable salt thereof, wherein X² does not contain a Q.

3. The compound of claim 1, or a pharmaceutically acceptable salt thereof, wherein X² does not contain a K or R.

4. The compound of claim 1, or a pharmaceutically acceptable salt thereof, wherein X² does not contain a Q, K, or R.

5. The compound of claim 1, or a pharmaceutically acceptable salt thereof, wherein X³ is G, A, V, I, or L.

6. The compound of claim 1, wherein the compound is of formula I, or a pharmaceutically acceptable salt thereof.

7. The compound of claim 1, wherein the compound is of formula II, or a pharmaceutically acceptable salt thereof.

8. The compound of claim 7, or a pharmaceutically acceptable salt thereof, wherein [symbol] represents —C(O)—O—, —C(O)—NH—, —NH—C(O)—, —S— 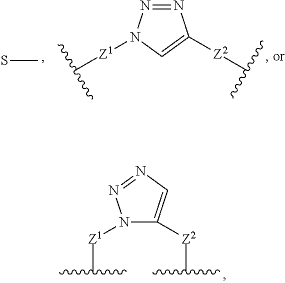
where Z¹ and Z² are independently selected —(CH₂)ₙ— groups, wherein n is an integer from 1 to 7, and wherein if n is an integer from 3 to 7, then one or two non-adjacent CH₂ groups may be replaced with groups independently selected from the group consisting of: —C(O)—, —NH—, —N(CH₃)—, —C(O)—NH—, —S—, —C(O)—O—, and —O—.

9. The compound of claim 1, or a pharmaceutically acceptable salt thereof, wherein G* is glycine.

10. The compound of claim 1, wherein said compound is selected from the group consisting of:
GAGDS (SEQ ID NO: 72);
GVGDS (SEQ ID NO: 73);
GIGDS (SEQ ID NO: 74); and
GLGDS (SEQ ID NO: 75); or a pharmaceutically acceptable salt thereof.

11. The compound of claim 1, or a pharmaceutically acceptable salt thereof, wherein the compound is of formula II, X¹ is mercaptopropionyl (Mpr) and Y¹ is absent.

12. The compound of claim 8, or a pharmaceutically acceptable salt thereof,
wherein [symbol] represents a disulfide bond,
wherein X¹ and X⁵ are independently selected from the group consisting of Mpr (mercaptopropionyl), Mvl (mercaptovaleryl), Cys, Pen (Penicillamine), Pmp (β₁,β-pentamethylene-β-mercaptopropionic acid), and Pmc (amino-β₁,β-pentamethylene-B-mercaptopropionic acid).

13. The compound of claim 12, or a pharmaceutically acceptable salt thereof, wherein X¹ is mercaptopropionyl (Mpr), X⁵ is Cys, and Y¹ is absent.

14. The compound of claim 1, or a pharmaceutically acceptable salt thereof, wherein Y² is NH₂.

15. The compound of claim 1, wherein G* is Glycine.

16. The compound of claim 5, wherein said compound is 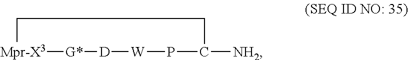
or a pharmaceutically acceptable salt thereof.

17. A pharmaceutical composition comprising:
a compound of claim 1, or a pharmaceutically acceptable salt thereof; and
a pharmaceutically acceptable carrier

18. A method of treating a platelet associated ischemic disorder in a patient comprising administering to said patient a therapeutically effective amount of a compound of claim 1, or a pharmaceutically acceptable salt thereof.

19. A method of treating platelet loss during extracorporeal circulation of blood comprising contacting said blood with a therapeutically effective amount of a compound of claim 1, or a pharmaceutically acceptable salt thereof.

20. A method of treating platelet aggregation, embolization or consumption of extracorporeal circulation comprising administering a therapeutically effective amount of a compound of claim 1, or a pharmaceutically acceptable salt thereof.

21. A method for inhibiting platelet aggregation in a mammal comprising administering a therapeutically effective amount of a compound of claim 1, or a pharmaceutically acceptable salt thereof.

22. A method for inhibiting clot formation in a mammal comprising administering an effective amount of a compound of claim 1, or a pharmaceutically acceptable salt thereof.

23. A method for treating myocardial infarction in a mammal comprising administering a therapeutically effective amount of a compound of claim 1, or a pharmaceutically acceptable salt thereof.

24. A method for treating stroke in a mammal comprising administering a therapeutically effective amount of a compound of claim 1, or a pharmaceutically acceptable salt thereof.

25. A method for treating patients with acute coronary syndrome comprising administering a therapeutically effective amount of a compound of claim 1, or a pharmaceutically acceptable salt thereof.


SEQUENCE LISTING (75 sequences)

SEQ ID NO: 1 - protein, 12 aa, Artificial Sequence
  peptide motif of the gamma chain of Fibrinogen
HHLGGAKQAGDV

SEQ ID NO: 2 - protein, 4 aa, Artificial Sequence
  synthesized peptide inhibitor
RGDS

SEQ ID NO: 3 - protein, 10 aa, Artificial Sequence
  gamma-derived peptide
LGGAKQAGDV

SEQ ID NO: 4 - protein, 5 aa, Artificial Sequence
  synthesized peptide inhibitor
GRGDS

SEQ ID NO: 5 - protein, 17 aa, Artificial Sequence
  linear platelet aggregation inhibitor
XXXXXXXXXDXXXXXXX

SEQ ID NO: 6 - protein, 17 aa, Artificial Sequence
  cyclic platelet aggregation inhibitor
XXXXXXXXXDXXXXXXX

SEQ ID NO: 7 - protein, 10 aa, Artificial Sequence
  cyclic platelet aggregation inhibitor
XXXXXXXXXD

SEQ ID NO: 8 - protein, 10 aa, Artificial Sequence
  cyclic platelet aggregation inhibitor
XXDXXXXXXX

SEQ ID NO: 9 - protein, 3 aa, Artificial Sequence
  cyclic platelet aggregation inhibitor
XXD

SEQ ID NO: 10 - protein, 6 aa, Artificial Sequence
  synthesized peptide inhibitor
LQAGDV

SEQ ID NO: 11 - protein, 6 aa, Artificial Sequence
  platelet aggregation inhibitor
GRGDSC

SEQ ID NO: 12 - protein, 5 aa, Artificial Sequence
  synthesized cyclic peptide
RGDFG

SEQ ID NO: 13 - protein, 7 aa, Artificial Sequence
  completely synthesized peptide
HHLGGAC

SEQ ID NO: 14 - protein, 7 aa, Artificial Sequence
  completely synthesized peptide
KQAGDVC

SEQ ID NO: 15 - protein, 7 aa, Artificial Sequence
  completely synthesized peptide
GGAKQAC

SEQ ID NO: 16 - protein, 6 aa, Artificial Sequence
  completely synthesized peptide
QAGDVC

SEQ ID NO: 17 - protein, 5 aa, Artificial Sequence
  completely synthesized peptide
AGDVC

SEQ ID NO: 18 - protein, 4 aa, Artificial Sequence
  completely synthesized peptide
GDVC

SEQ ID NO: 19 - protein, 6 aa, Artificial Sequence
  completely synthesized peptide
KQAGDC

SEQ ID NO: 20 - protein, 5 aa, Artificial Sequence
  completely synthesized peptide
KQAGC

SEQ ID NO: 21 - protein, 13 aa, Artificial Sequence
  platelet aggregation inhibitor
HHLGGAKQAGDVC

SEQ ID NO: 22 - protein, 4 aa, Artificial Sequence
  completely synthesized peptide
AGDV

SEQ ID NO: 23 - protein, 6 aa, Artificial Sequence
  scrambled peptide
GGRDGS

SEQ ID NO: 24 - protein, 13 aa, Artificial Sequence
  scrambled peptide
GHHLGGADQAGKV

SEQ ID NO: 25 - protein, 5 aa, Artificial Sequence
  scrambled peptide
GADGV

SEQ ID NO: 26 - protein, 6 aa, Artificial Sequence
  platelet aggregation inhibitor
GXGDSC

SEQ ID NO: 27 - protein, 6 aa, Artificial Sequence
  completely synthesized peptide
GXGDSC

SEQ ID NO: 28 - protein, 6 aa, Artificial Sequence
  platelet aggregation inhibitor
GRGDSC

SEQ ID NO: 29 - protein, 6 aa, Artificial Sequence
  completely synthesized peptide
GGGDSC

SEQ ID NO: 30 - protein, 6 aa, Artificial Sequence
  completely synthesized peptide
GAGDSC

SEQ ID NO: 31 - protein, 6 aa, Artificial Sequence
  completely synthesized peptide
GKGDSC

SEQ ID NO: 32 - protein, 6 aa, Artificial Sequence
  completely synthesized peptide
GVGDSC

SEQ ID NO: 33 - protein, 6 aa, Artificial Sequence
  completely synthesized peptide
GIGDSC

SEQ ID NO: 34 - protein, 6 aa, Artificial Sequence
  platelet aggregation inhibitor
GLGDSC

SEQ ID NO: 35 - protein, 7 aa, Artificial Sequence
  cyclic S-S platelet aggregation inhibitor
XXGDWPC

SEQ ID NO: 36 - protein, 7 aa, Artificial Sequence
  cyclic S-S platelet aggregation inhibitor
XAGDWPC

SEQ ID NO: 37 - protein, 7 aa, Artificial Sequence
  cyclic S-S platelet aggregation inhibitor
XVGDWPC

SEQ ID NO: 38 - protein, 7 aa, Artificial Sequence
  cyclic S-S platelet aggregation inhibitor
XLGDWPC

SEQ ID NO: 39 - protein, 7 aa, Artificial Sequence
  cyclic S-S platelet aggregation inhibitor
XIGDWPC

SEQ ID NO: 40 - protein, 7 aa, Artificial Sequence
  cyclic S-S platelet aggregation inhibitor
XGGDWPC

SEQ ID NO: 41 - protein, 7 aa, Artificial Sequence
  cyclic (C(O)-NH) platelet aggregation inhibitor
GGXGDWP

SEQ ID NO: 42 - protein, 7 aa, Artificial Sequence
  cyclic (C(O)-NH) platelet aggregation inhibitor
GGAGDWP

SEQ ID NO: 43 - protein, 7 aa, Artificial Sequence
  cyclic (C(O)-NH) platelet aggregation inhibitor
GGLGDWP

SEQ ID NO: 44 - protein, 7 aa, Artificial Sequence
  cyclic (C(O)-NH) platelet aggregation inhibitor
GGGGDWP

SEQ ID NO: 45 - protein, 7 aa, Artificial Sequence
  cyclic (C(O)-NH) platelet aggregation inhibitor
GGVGDWP

SEQ ID NO: 46 - protein, 7 aa, Artificial Sequence
  cyclic (C(O)-NH) platelet aggregation inhibitor
GGIGDWP

SEQ ID NO: 47 - protein, 7 aa, Artificial Sequence
  cyclic platelet aggregation inhibitor
GGXGDWP

SEQ ID NO: 48 - protein, 7 aa, Artificial Sequence
  cyclic platelet aggregation inhibitor
GGAGDWP

SEQ ID NO: 49 - protein, 7 aa, Artificial Sequence
  cyclic platelet aggregation inhibitor
GGAGDXP

SEQ ID NO: 50 - protein, 7 aa, Artificial Sequence
  cyclic platelet aggregation inhibitor
GGAGDWP

SEQ ID NO: 51 - protein, 7 aa, Artificial Sequence
  cyclic (C(O)-NH) platelet aggregation inhibitor
GXAGDWP

SEQ ID NO: 52 - protein, 7 aa, Artificial Sequence
  cyclic (C(O)-NH) platelet aggregation inhibitor
GWAGDWP

SEQ ID NO: 53 - protein, 7 aa, Artificial Sequence
  cyclic (C(O)-NH) platelet aggregation inhibitor
GEAGDWP

SEQ ID NO: 54 - protein, 7 aa, Artificial Sequence
  cyclic (C(O)-NH) platelet aggregation inhibitor
GRAGDWP

SEQ ID NO: 55 - protein, 7 aa, Artificial Sequence
  cyclic (C(O)-NH) platelet aggregation inhibitor
GHAGDWP

SEQ ID NO: 56 - protein, 7 aa, Artificial Sequence
  cyclic (C(O)-NH) platelet aggregation inhibitor
GGAGDWP

SEQ ID NO: 57 - protein, 7 aa, Artificial Sequence
  cyclic (C(O)-NH) platelet aggregation inhibitor
GSAGDWP

SEQ ID NO: 58 - protein, 7 aa, Artificial Sequence
  cyclic (C(O)-NH) platelet aggregation inhibitor
GGAGDWP

SEQ ID NO: 59 - protein, 6 aa, Artificial Sequence
  cyclic (C(O)-NH) platelet aggregation inhibitor
GAGDWP

SEQ ID NO: 60 - protein, 6 aa, Artificial Sequence
  cyclic (C(O)-NH) platelet aggregation inhibitor
GGAGDW

SEQ ID NO: 61 - protein, 5 aa, Artificial Sequence
  cyclic (C(O)-NH) platelet aggregation inhibitor
GAGDW

SEQ ID NO: 62 - protein, 5 aa, Artificial Sequence
  cyclic (C(O)-NH) platelet aggregation inhibitor
GGAGD

SEQ ID NO: 63 - protein, 5 aa, Artificial Sequence
  cyclic (C(O)-NH) platelet aggregation inhibitor
GGVGD

SEQ ID NO: 64 - protein, 5 aa, Artificial Sequence
  cyclic (C(O)-NH) platelet aggregation inhibitor
GGIGD

SEQ ID NO: 65 - protein, 5 aa, Artificial Sequence
  cyclic (C(O)-NH) platelet aggregation inhibitor
GGLGD

SEQ ID NO: 66 - protein, 5 aa, Artificial Sequence
  cyclic (C(O)-NH) platelet aggregation inhibitor
GGGGD

SEQ ID NO: 67 - protein, 5 aa, Artificial Sequence
  cyclic (C(O)-NH) platelet aggregation inhibitor
AGDWP

SEQ ID NO: 68 - protein, 4 aa, Artificial Sequence
  cyclic (C(O)-NH) platelet aggregation inhibitor
AGDW

SEQ ID NO: 69 - protein, 3 aa, Artificial Sequence
  cyclic (C(O)-NH) platelet aggregation inhibitor
AGD

SEQ ID NO: 70 - protein, 7 aa, Artificial Sequence
  scrambled peptide
GGRDGSC

SEQ ID NO: 71 - protein, 14 aa, Artificial Sequence
  scrambled peptide
GHHLGGADQAGKVC

SEQ ID NO: 72 - protein, 5 aa, Artificial Sequence
  completely synthesized peptide
GAGDS

SEQ ID NO: 73 - protein, 5 aa, Artificial Sequence
  completely synthesized peptide
GVGDS

SEQ ID NO: 74 - protein, 5 aa, Artificial Sequence
  completely synthesized peptide
GIGDS

SEQ ID NO: 75 - protein, 5 aa, Artificial Sequence
  completely synthesized peptide
GLGDS